(19) United States
(12) Patent Application Publication
(10) Pub. No.: US 2020/0038227 A1
(43) Pub. Date: Feb. 6, 2020

(54) COLOSTOMY BAG SPACER ASSEMBLY

(71) Applicant: Thomas Makar, JR., Warrenton, VA (US)

(72) Inventor: Thomas Makar, JR., Warrenton, VA (US)

(21) Appl. No.: 16/050,665

(22) Filed: Jul. 31, 2018

(51) Int. Cl. A61F 5/44; A61F 5/445

(52) U.S. Cl. CPC A61F 5/4404; A61F 5/445

(57) ABSTRACT

A colostomy bag spacer assembly for reducing leakage of an ostomy bag includes an ostomy bag that has an input port. An ostomy wafer is included that has an aperture extending therethrough for insertably receiving a stoma. The ostomy wafer includes a retainer that is coupled thereto. A spacer is releasably coupled to the retainer and the input port on the ostomy bag insertably receives the spacer. Thus, the ostomy bag is spaced from the user's skin to resist being compressed against the user's skin. In this way feces is inhibited from leaking between the ostomy wafer and the user's skin.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to spacer devices and more particularly pertains to a new spacer device for reducing leakage of an ostomy bag.

### BRIEF SUMMARY OF THE INVENTION

An embodiment of the disclosure meets the needs presented above by generally comprising an ostomy bag that has an input port. An ostomy wafer is included that has an aperture extending therethrough for insertably receiving a stoma. The ostomy wafer includes a retainer that is coupled thereto. A spacer is releasably coupled to the retainer and the input port on the ostomy bag insertably receives the spacer. Thus, the ostomy bag is spaced from the user's skin to resist being compressed against the user's skin. In this way feces is inhibited from leaking between the ostomy wafer and the user's skin.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a colostomy bag spacer assembly according to an embodiment of the disclosure.

FIG. 2 is a right side view of a spacer of an embodiment of the disclosure.

FIG. 3 is a bottom view of a spacer of an embodiment of the disclosure.

FIG. 4 is an exploded perspective view of an embodiment of the disclosure.

FIG. 5 is a perspective in-use view of an embodiment of the disclosure.

FIG. 6 is a cross sectional view taken along line 6-6 of FIG. 1 of an embodiment of the disclosure.

### DETAILED DESCRIPTION OF THE INVENTION

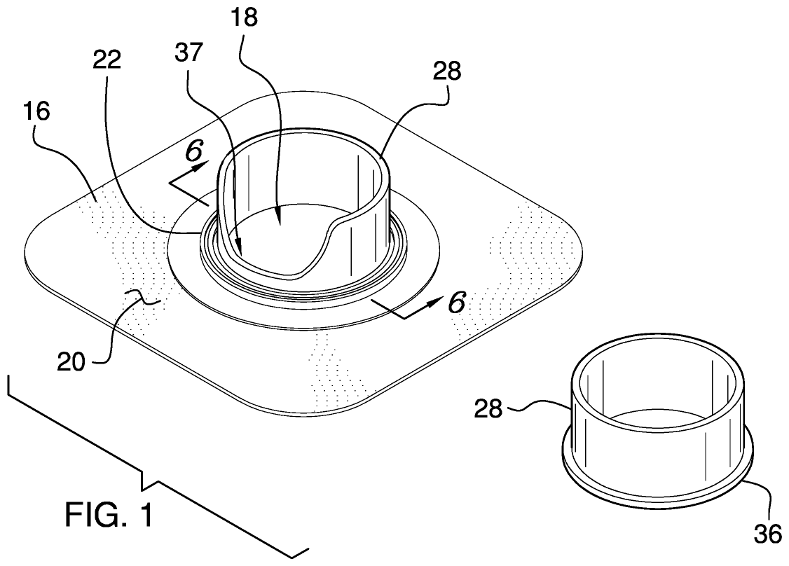

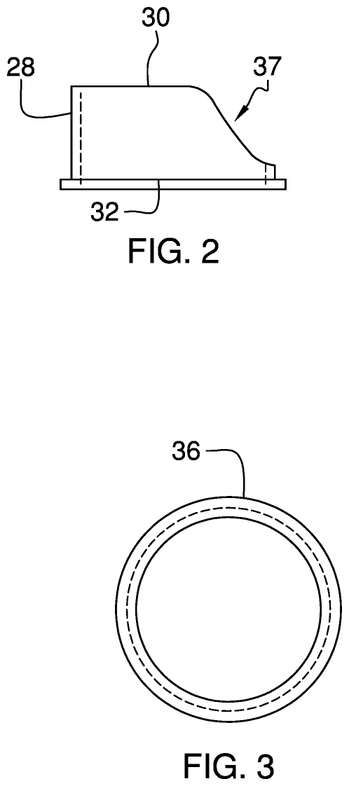

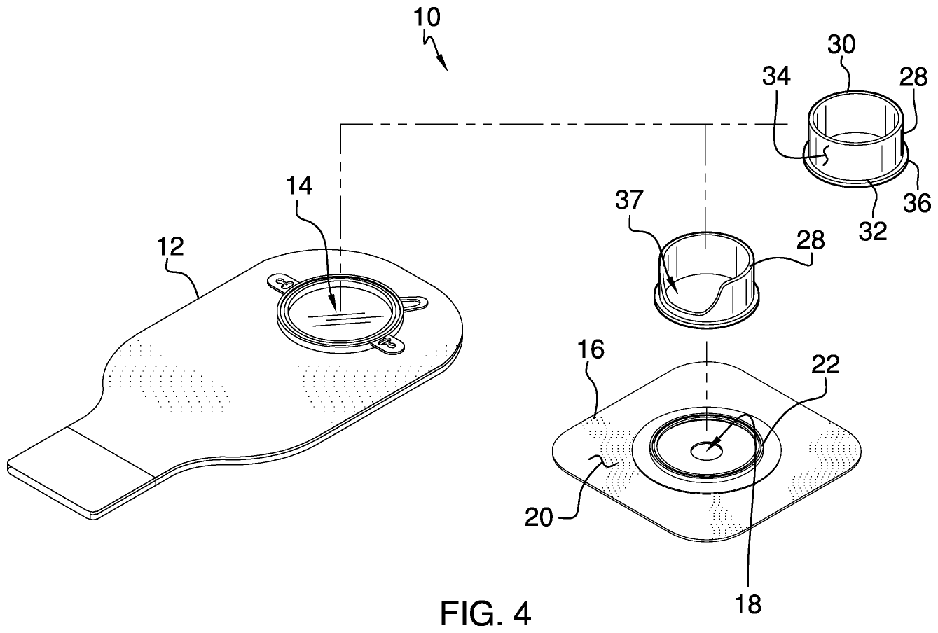

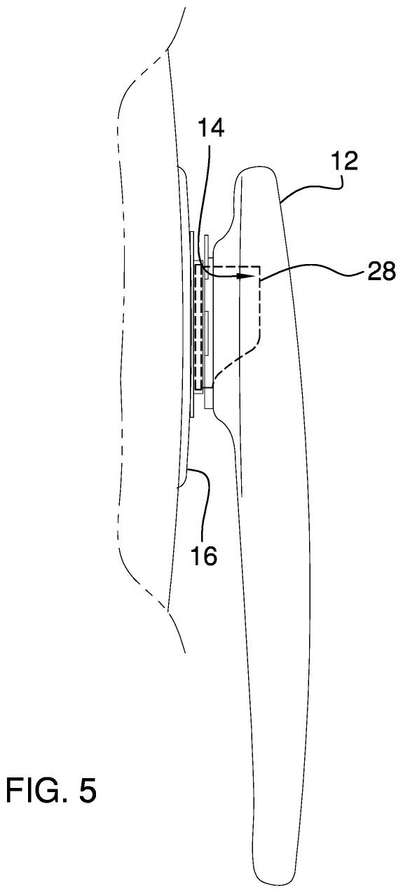

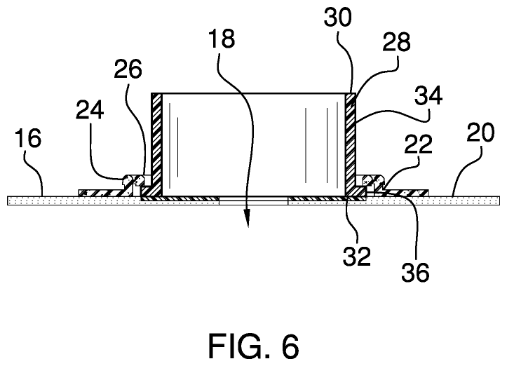

With reference now to the drawings, and in particular to FIGS. 1 through 6 thereof, a new spacer device embodying the principles and concepts of an embodiment of the disclosure and generally designated by the reference numeral 10 will be described.

As best illustrated in FIGS. 1 through 6, the colostomy bag spacer assembly 10 generally comprises an ostomy bag 12 that has an input port 14. The ostomy bag 12 may be an ostomy bag of any conventional design, such as that used for a colostomy or the like. An ostomy wafer 16 is included and the ostomy wafer 16 has an aperture 18 extending therethrough for insertably receiving a stoma. The ostomy wafer 16 may be an ostomy wafer of any conventional design, such as those used for a colostomy bag or the like.

The ostomy wafer 16 has a first surface 20 and the ostomy wafer 16 includes a retainer 22 that is coupled thereto. The retainer 22 is continuously arcuate such that the retainer 22 defines a ring. Moreover, the retainer 22 comprises a leg 24 extending away from the first surface 20 of the ostomy wafer 16 and a foot 26 that is spaced from the first surface 20, and the retainer 22 is positioned to surround the aperture 18.

A spacer 28 is provided and the spacer 28 is releasably coupled to the retainer 22. The input port 14 on the ostomy bag 12 insertably receives the spacer 28 such that the ostomy bag 12 is spaced from the user's skin. In this way the ostomy bag 12 resists being compressed against the user's skin thereby inhibiting leakage of feces around the ostomy wafer 16. Thus, a toddler or infant that has a colostomy bag 12 can play and crawl without compressing the colostomy bag 12 against themselves and potentially causing leakage between the wafer and the toddler or infant's skin.

The spacer 28 has a top edge 30, a bottom edge 32 and an outer surface 34 extending therebetween, and the outer surface 34 is continuously arcuate. The spacer 28 may have a height ranging between approximately 15.0 mm and 20.0 mm and a diameter ranging between approximately 44.0 mm and 48.0 mm. Moreover, the spacer 28 may be constructed to conform to any design and size of ostomy wafer 16. A lip 36 is coupled to the spacer 28 and the lip 36 engages the retainer 22 thereby retaining the spacer 28 on the wafer. The top edge 30 of the spacer 28 may have a cut out portion 37 curving downwardly toward the bottom edge 32. The cut out portion 37 may be directed downwardly in the ostomy bag 12 for directing the feces downwardly into the ostomy bag 12.

The lip 36 extends outwardly from the outer surface 34 of the spacer 28, and the lip 36 is aligned with and is coextensive with the bottom edge 32. The lip 36 is positioned between the foot 26 on the retainer 22 and the first surface 20 of the wafer when the spacer 28 is releasably coupled to the retainer 22. In this way the spacer 28 is releasably coupled to the wafer and the lip 36 forms a fluid impermeable seal with the retainer 22. Additionally, the input port 14 on the ostomy bag 12 insertably receives the top edge 30 of the spacer 28. A bounding edge 38 of the input port 14 frictionally engages the outer surface 34 of the spacer 28 to inhibit feces from leaking between the spacer 28 and the input port 14.

In use, the lip 36 on the spacer 28 is inserted between the foot 26 on the retainer 22 and the first surface 20 of the ostomy wafer 16. In this way the spacer 28 is coupled to the ostomy wafer 16. The ostomy bag 12 is coupled to the spacer 28 having the input port 14 insertably receiving the spacer 28. Thus, the ostomy bag 12 is spaced from the user's skin rather than lying against the user's skin as would be the case in a conventional application of the ostomy bag 12. In this way the ostomy bag 12 can be employed on a child who may potentially compress the ostomy bag 12 against themselves while playing or crawling. Conventional ostomy bags 12 cause feces to leak between the ostomy wafer 16 and the user's skin when the ostomy bag 12 becomes compressed against the user's skin. The spacer 28 serves to inhibit the ostomy bag 12 from becoming compressed, thereby reducing the likelihood of leakage occurring between the ostomy wafer 16 and the user's skin.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A colostomy bag spacer assembly being configured to space a colostomy bag from a user's skin thereby inhibiting said colostomy bag from pancaking, said assembly comprising:
an ostomy bag having an input port;
an ostomy wafer having an aperture extending therethrough for insertably receiving a stoma, said ostomy wafer including a retainer being coupled thereto; and
a spacer being releasably coupled to said retainer, said input port on said ostomy bag insertably receiving said spacer such that said ostomy bag is spaced from the user's skin to resist being compressed against the user's skin thereby inhibiting leakage of feces between said ostomy wafer and the user's skin.

2. The assembly according to claim 1, wherein:
said retainer is continuously arcuate such that said retainer defines a ring, said retainer comprising a leg extending away from said first surface of said ostomy wafer and a foot being spaced from said first surface, said retainer being positioned to surround said aperture; and
said spacer has a top edge, a bottom edge and an outer surface extending therebetween, said outer surface being continuously arcuate.

3. The assembly according to claim 2, further comprising a lip being coupled to said spacer, said lip engaging said retainer thereby retaining said spacer on said wafer said lip extending outwardly from said outer surface of said spacer, said being aligned with and being coextensive with said bottom edge, said lip being positioned between a foot on said retainer and a first surface of said wafer when said spacer is releasably coupled to said retainer.

4. The assembly according to claim 2, wherein said top edge of said spacer has a cut out portion curving downwardly toward said bottom edge.

5. The assembly according to claim 3, wherein said input port on said ostomy bag insertably receives said top edge of said spacer having a bounding edge of said input port frictionally engaging said outer surface of said spacer.

6. A colostomy bag spacer assembly being configured to space a colostomy bag from a user's skin thereby inhibiting said colostomy bag from pancaking, said assembly comprising:
an ostomy bag having an input port;
an ostomy wafer having an aperture extending therethrough for insertably receiving a stoma, said ostomy wafer having a first surface, said ostomy wafer including a retainer being coupled thereto, said retainer being continuously arcuate such that said retainer defines a ring, said retainer comprising a leg extending away from said first surface of said ostomy wafer and a foot being spaced from said first surface, said retainer being positioned to surround said aperture;
a spacer being releasably coupled to said retainer, said input port on said ostomy bag insertably receiving said spacer such that said ostomy bag is spaced from the user's skin to resist being compressed against the user's skin thereby inhibiting leakage of feces between said ostomy wafer and the user's skin, said spacer having a top edge, a bottom edge and an outer surface extending therebetween, said outer surface being continuously arcuate;
a lip being coupled to said spacer, said lip engaging said retainer thereby retaining said spacer on said wafer, said lip extending outwardly from said outer surface of said spacer, said being aligned with and being coextensive with said bottom edge, said lip being positioned between said foot on said retainer and said first surface of said wafer when said spacer is releasably coupled to said retainer, said input port on said ostomy bag insertably receiving said top edge of said spacer having a bounding edge of said input port frictionally engaging said outer surface of said spacer.

7. The assembly according to claim 6, wherein said top edge has a cut out portion curving downwardly toward said bottom edge.
